(19) United States
(12) Patent Application Publication
(10) Pub. No.: US 2001/0023503 A1
(43) Pub. Date: Sep. 27, 2001

(54) Optical diving mask

(76) Inventor: Giovanni Garofalo, Rapallo (IT)

Correspondence Address: LARSON & TAYLOR, PLC, 1199 NORTH FAIRFAX STREET, SUITE 900, ALEXANDRIA, VA, 22314, US

(21) Appl. No.: 09/737,856

(22) Filed: Dec. 18, 2000

(30) Foreign Application Priority Data
Jan. 5, 2000 (IT) GE2000 U 000001

(51) Int. Cl. A61F 9/02

(52) U.S. Cl. 2/439

(57) ABSTRACT

Diving mask, in particular proper to fit optical glasses, including a face of elastomeric material, provided with at least one opening, a stiff body having at least one seat to house a glass or lens. Watertight means are provided between said lens or glass and said seat, and means means are provided for holding said lens or glass in said seat. Along a substantial part of the perimetric edge of said stiff body, on a plane substantially parallel to the lying one of the lens or glass, guiding means are formed for the insertion of the holding means of the lens or glass into said housing.

### BACKGROUND AND SUMMARY OF THE INVENTION

The present invention refers to diving masks, and in particular refers to diving masks where it is possible to use graduated lens instead of normal glass.

A problem present since years in the production of diving masks is concerned with the possibility to make them suitable to fit both tempered not graduated lens and corrective lens, to help those users needing to use such a correction. In many cases the most common way consisted of complicated interventions both by specialized opticians and the maker himself. However this kind of changes could produce masks provided with fixed fitted lens, and then not adjustable in case of eventual changes in the user visual defect.

An interesting solution to this kind of problem is offered by the mask described in the U.S. Pat. No. 4,056,853.

Said mask is provided with a quite stiff plastic body where are obtained two seats proper to house two lens; around the two seats are two U shaped relief edges opened to the side ends of the mask; in the edges are obtained two grooves where are housed two suitable frames sliding within the same ones and provided with locking means.

An evident problem is due to the impossibility to differ in the embodiment of the mask from a certain outline provided for the molding of the stiff body; the user choice of the shape of the mask is then strictly limited, in particular this system appears to be little or not at all suitable for monocular type masks. Moreover, the assembly described above is difficult to use, and even its embodiment seems to be not very easy.

Aim of the present invention is then to provide a diving mask, in particular proper to fit optical glasses, easy in its embodiment and using and allowing a large choice among different shapes.

Object of the present invention is then a diving mask, in particular proper to fit optical glasses, including a face of elastomeric material, provided with at least one opening, a stiff body where is at least one housing seat for a glass or lens, being provided watertight means between said lens or glass and said seat, and holding means for said lens or glass in said seat; characterized in that along a substantial part of the perimetric edge of said stiff body are obtained, on a plane substantially parallel to the one of the lens or glass, guiding means for the insertion of the holding means of the lens or glass into said seat.

Said guiding means can include a groove obtained on the wall turned to the inside of the stiff body of a relief axially projecting from the perimetric edge of the same. Advantageously, said relief and said groove extend substantially for the half of the stiff body perimeter, and in particular for the half turned to the diver's mouth.

The holding means can include a stiff frame, in plastic or metallic material, being provided means to lock the coupling between said frame and said stiff body.

### BRIEF DESCRIPTION OF THE DRAWINGS

Further advantages and features of the present invention will be better understood from the following detailed description of an illustrative embodiment form of the same, not to be considered as a limitative description, with reference to the enclosed drawings, where:

FIG. 1 is a front elevation view with exploded parts of an embodiment form of the mask according to the present invention;

FIG. 2 is an elevation view of the mask shown in FIG. 1, fully assembled; and

FIG. 3 is a section view along the line III-III of FIG. 2.

### DESCRIPTION OF THE PREFERRED EMBODIMENT OF THE INVENTION

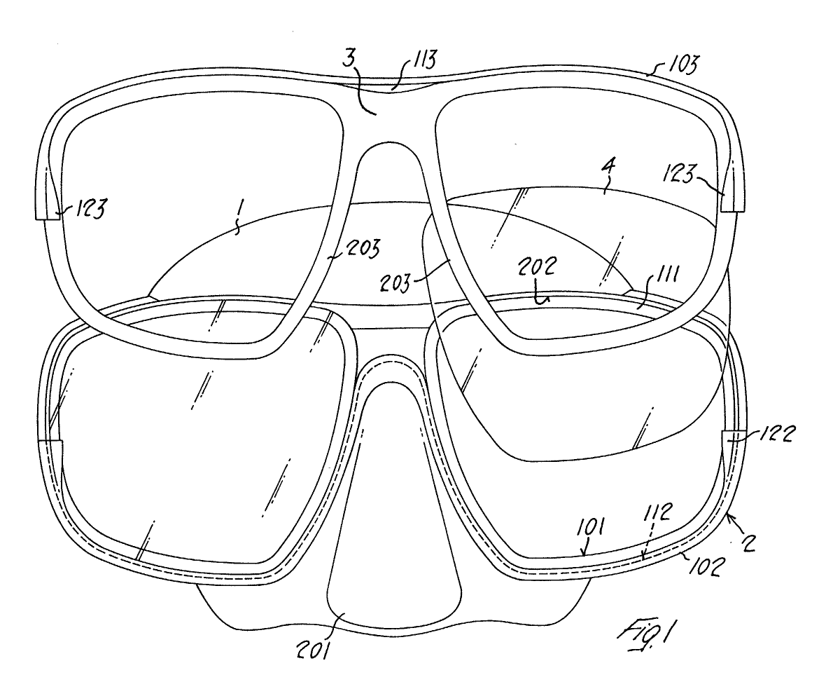

In FIG. 1 is shown a mask according to the present invention; with 1 is indicated the face of said mask, provided in the shown case with two openings 101 corresponding to the two ocular elements, it is to say the two glasses 4, and the compartment 201 housing the diver's nose. The mask includes a stiff body 2, normally made of thermoplastic material, where area obtained the two seats 202 apt to house the glasses 4; in the seats 202 are set the watertight elements 111, obtained from the same piece of the face 1.

Along the perimetric edge of the stiff body 2, in the part turned to the compartment 201 housing the diver's nose, is obtained a relief 102 axially projecting from said body 2, where it is obtained, on the wall turned to the inside, a groove 112. In said groove 112 it is inserted the frame 3, apt to keep the glasses 4 in contact with the watertight elements 111 in the seats 202. The frame 3 is provided, on the edge opposite the one destined to cooperate with the groove 112 of the relief 102, with a framework 103 integral with said relief 102, to which is connected through the insertion into cavities obtained in slubs 113 and 123.

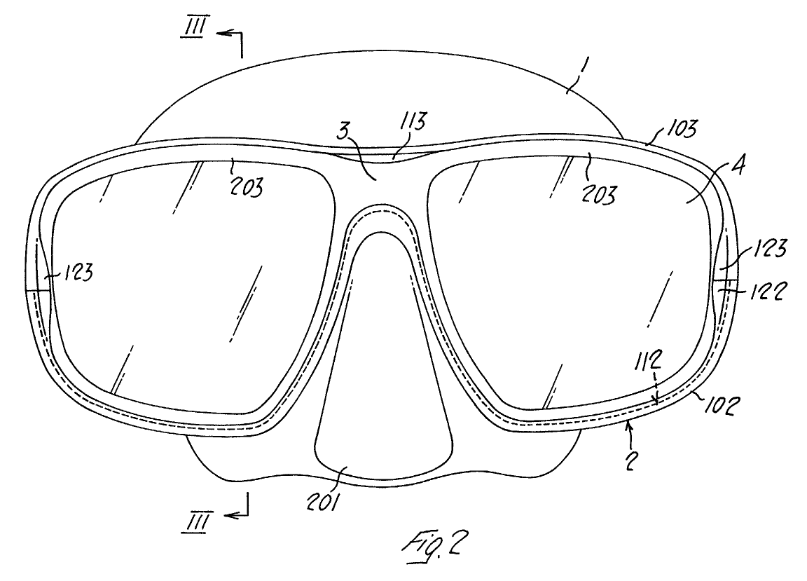

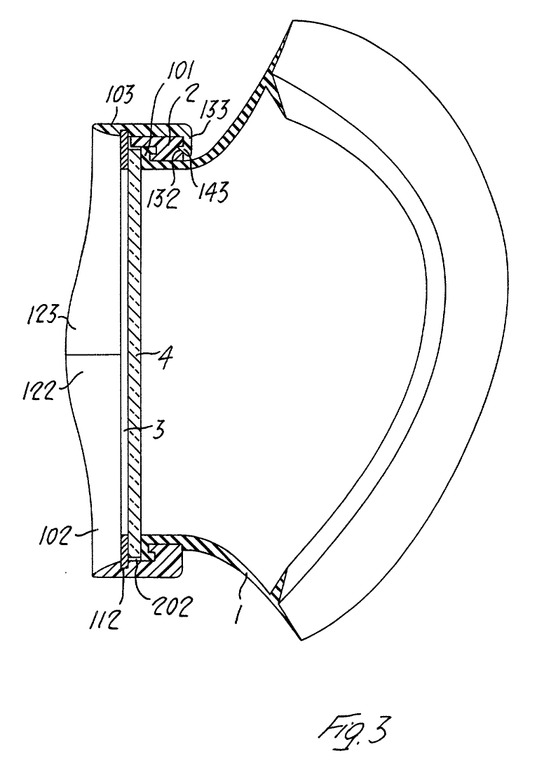

As appearing from FIG. 2, a substantial part of the frame 3 edge 203 is housed in the groove 112 of the relief 102 projecting from the body 2. In FIG. 3 is finally shown the section along the line III-III of FIG. 2; in the part of the frame 103 turned to the face 1 there is a fin 133 bent to the body 2, whose free end is a tooth relief 143, co-operating with a complementary cavity 132 obtained in the body 2. In the figure is moreover marked the action of the frame 3 on the edge of the glasses 4, which is brought into contact with the watertight elements 111 in the housings 202.

The functioning of the mask according to the present invention will be better understood from what follows. In FIG. 1 the mask is shown during the assembling; one of the two glasses 4 has already been inserted in the suitable hollow seat in the body 2; in the seat 202 the watertight is guaranteed by the element 111, which is obtained from the same piece of the mask face 1. As emerges from FIG. 3, the large contact surface between the glass 4 and the element 111 guarantees a good sealing. Then it is inserted the frame 3, whose edges 203 are positioned into the groove 112. The part of the frame not inserted in the groove 112 is coupled to the framework 103, which carries the connection means with the body 2, it is to say one or more fins 133 provided with the tooth relief 143 co-operating with the cavities 132 obtained on the body 2 itself.

It appears clear that this kind of solution allows absolute freedom from the point of view of the choice of the shapes of the stiff body. In fact, the groove 112 is obtained in the inner wall of the relief 102 projecting from the body 2 perimetric edge, then for any different perimeter it will be a different groove to house a suitably shaped frame, while the mask described in the US patent mentioned above has a practically compulsory shape. Moreover, even if in this case it is shown a two oculars mask, this kind of embodiment is suitable for one ocular masks as well.

Also advantageous is to use just one frame to hold both the glasses, or lens, in the case of two oculars as shown; the use appears extremely easier for the user and the glasses or lens changing operations are very easy and quick.

The mask so devised makes much more easy the use of optical lens or optically corrected glasses, without particularly influencing the choice of the shape of the mask itself.

I claim:

1. A diving mask, particularly apt to accomodate optical glasses, comprising a face piece of elastomeric material, provided with at least one opening, a stiff body having at least one seat to house a glass or lens; watertight means between said lens or glass and said seat, holding means for holding said lens or glass in said seat; and guiding means formed along a substantial part of the perimetric edge of said stiff body, on a plane substantially parallel to the lying plane one of the said lens or glass, for the insertion of the holding means of said lens or glass into said seat.

2. A diving mask according to claim 1, where said guiding means include a groove obtained on the wall turned to the inside of the stiff body of a relief axially projecting from the perimetric edge of the same.

3. A diving mask according to claim 2, where said relief and said groove project substantially for the half of the stiff body perimeter, and preferably along the side turned to the diver's mouth.

4. A mask according to claim 1, where the said lens or glass holding means in said seat include a stiff frame, of plastic or metallic material, shaped so to follow the perimeter edge of said glass or lens, being provided locking means of the coupling between said frame and said stiff body.

5. A diving mask according to claim 4, where said frame is provided, along the edge part not destined to the insertion of said guiding means, with a framework provided with one or more fins bent to the face of said mask and each one provided at its free end with a tooth relief co-operating with a suitable cavity obtained in said stiff body.

6. A diving mask according to claim 5, where said framework includes one or more slubs provided with a cavity for the housing of said frame.

7. A diving mask according to claim 1, where said watertight elements are obtained from the same piece of said face.
